SEQUENCE LISTING (9 sequences)

SEQ ID NO: 1 - protein, 16 aa, Homo sapiens
ANTPCGPYTHDCPVKR

SEQ ID NO: 2 - protein, 122 aa, Homo sapiens
MCTCSPSHPQDAFCNSDIVIRAKVVGKKLVKEGPFGTLVYTIKQMKMYRGFTKMPHVQYIHTEASESLCGLKLEVNKYQYLLTGRVYDGKMYTGLCNFVERWDQLTLSQRKGLNYRYHLGCK

SEQ ID NO: 3 - protein, 138 aa, Homo sapiens
ANTPCGPYTHDCPVKRMCTCSPSHPQDAFCNSDIVIRAKVVGKKLVKEGPFGTLVYTIKQMKMYRGFTKMPHVQYIHTEASESLCGLKLEVNKYQYLLTGRVYDGKMYTGLCNFVERWDQLTLSQRKGLNYRYHLGCK

SEQ ID NO: 4 - protein, 138 aa, Homo sapiens
ANTPCGPYTHDCPVKRMCGCSPSHPQDAFCNSDIVIRAKVVGKKLVKEGPFGTLVYTIKQMKMYRGFTKMPHVQYIHTEASESLCGLKLEVNKYQYLLTGRVYDGKMYTGLCNFVERWDQLTLSQRKGLNYRYHLGCK

SEQ ID NO: 5 - protein, 139 aa, Homo sapiens
ANTPCGPYTHDCPVKRMACTCSPSHPQDAFCNSDIVIRAKVVGKKLVKEGPFGTLVYTIKQMKMYRGFTKMPHVQYIHTEASESLCGLKLEVNKYQYLLTGRVYDGKMYTGLCNFVERWDQLTLSQRKGLNYRYHLGCK

SEQ ID NO: 6 - protein, 16 aa, Homo sapiens
KKEEEKKEEEKKEEEK

SEQ ID NO: 7 - protein, 138 aa, Homo sapiens
KKEEEKKEEEKKEEEKMCTCSPSHPQDAFCNSDIVIRAKVVGKKLVKEGPFGTLVYTIKQMKMYRGFTKMPHVQYIHTEASESLCGLKLEVNKYQYLLTGRVYDGKMYTGLCNFVERWDQLTLSQRKGLNYRYHLGCK

SEQ ID NO: 8 - protein, 138 aa, Homo sapiens
KKEEEKKEEEKKEEEKMCGCSPSHPQDAFCNSDIVIRAKVVGKKLVKEGPFGTLVYTIKQMKMYRGFTKMPHVQYIHTEASESLCGLKLEVNKYQYLLTGRVYDGKMYTGLCNFVERWDQLTLSQRKGLNYRYHLGCK

SEQ ID NO: 9 - protein, 139 aa, Homo sapiens
KKEEEKKEEEKKEEEKMACTCSPSHPQDAFCNSDIVIRAKVVGKKLVKEGPFGTLVYTIKQMKMYRGFTKMPHVQYIHTEASESLCGLKLEVNKYQYLLTGRVYDGKMYTGLCNFVERWDQLTLSQRKGLNYRYHLGCK

(19) United States
(12) Patent Application Publication
(10) Pub. No.: US 2021/0115111 A1
(43) Pub. Date: Apr. 22, 2021

(54) USE OF A PEPTIDE DERIVED FROM THE HUMAN PROTEIN NTIMP3 IN THE TREATMENT OF DIABETIC NEPHROPATHY

(71) Applicants: UNIVERSITA' DEGLI STUDI DI ROMA "TOR VERGATA", Roma (IT); UNIVERSITA' DEGLI STUDI DI ROMA "LA SAPIENZA", Rome (IT)

(72) Inventors: Massimo FEDERICI, Rome (IT); Rossella MENGHINI, Roma (IT); Viviana CASAGRANDE, Pomezia (RM) (IT); Stefano MENINI, Genova (IT)

(21) Appl. No.: 16/964,071

(22) PCT Filed: Jan. 21, 2019
(86) PCT No.: PCT/IB2019/050482

§ 371 Date: Jul. 22, 2020

(30) Foreign Application Priority Data
Jan. 23, 2018 (IT) 102018000001663

(51) Int. Cl. C07K 14/81; A61K 38/00; A61P 13/12

(52) U.S. Cl. CPC C07K 14/8146; C07K 2319/33; A61P 13/12; A61K 38/00

(57) ABSTRACT

Disclosed are fusion peptides consisting of the peptide fragment corresponding to the N-terminal domain derived from the human TIMP3 protein, both in native and mutated form, bound by the N-terminal end to a highly selective and efficient carrier peptide for transport in renal proximal tubule cells, the medical use thereof, in particular the use thereof in the treatment of diabetic nephropathy, and the compositions comprising them.

### INCORPORATION BY REFERENCE

The text file named Final SEQ ID, created on Jan. 8, 2021, and sized 8,998 bytes, which contains sequence ID listings, is herein expressly incorporated by reference.

### SCOPE OF THE INVENTION

The present invention relates to the field of the treatment of renal pathology and, more specifically, it relates to a peptide derivative of the human protein TIMP3, capable of restoring a high activity of the protein directly at the renal level in conditions, such as diabetic nephropathy, where a reduction thereof is related to the disease.

### BACKGROUND ART

Diabetic nephropathy (DN) is one of the most serious complications associated with type 1 and type 2 diabetes, which occurs in about a third of diabetic patients [Groop et al. “The presence and severity of chronic kidney disease predicts all-cause mortality in type 1 diabetes”. 2009; Diabetes. 58: 1651-8]. The condition is characterized by albuminuria, glomerulosclerosis and progressive loss of renal function exacerbated by metabolic and hemodynamic alterations in diabetes. The disease deteriorates in a rather slow but irreversible manner the renal function in diabetic patients, especially those in which the disease has existed for many years. The clinically established form generally appears about 15-25 years after the onset of diabetes.

In addition to the typical slow and gradual decline in renal function, with a tendency to proteinuria and renal failure, other clinical features characteristic of the condition are persistent microalbuminuria (between 50 and 300 mg/day) and arterial hypertension, resulting in a high risk of cardiovascular morbidity and mortality.

The accumulation of extracellular matrix in the glomerular basement membrane is a cyto-histological characteristic of the condition, which suggests a possible involvement of matrix metalloproteases in the development of diabetic nephropathy.

Recently, a cross-talk between angiotensin II (ATII) and the epidermal growth factor receptor (EGFR) has been reported, which appears to play a role in the development of renal lesions. Angiotensin II is also responsible for the redistribution of the ADAM17 metalloproteases to the apical membrane of the renal tubules [Lautrette A. et al. “Angiotensin II and EGF receptor cross-talk in chronic kidney diseases: a new therapeutic approach” Nat Med. 2005; 11: 867-74.

The ADAM17 metalloprotease belongs to the family of ADAM proteins, a family of transmembrane glycoproteins characterized by a multi-domain structure, including a pro-peptide domain that maintains the metalloprotease in an inactive state and must be removed before the enzyme is activated, a catalytic domain, a disintegrin domain. These enzymes are inhibitors of matrix metalloproteases, a group of peptidases involved in the degradation of the extracellular matrix (ECM). Protein expression is induced in response to mitogenic stimulation. The majority of ADAM proteins intervene in cell-cell fusion and cellular signaling processes, intervene in the continuous remodeling of the extracellular matrix and in the cleavage of the cell surface proteins [Dreymueller D. et al. “The role of ADAM-mediated shedding in vascular biology”. Eur J Cell Biol. 2012; 91: 472-85].

So far, 31 proteins have been identified that can be traced back to ADAM metalloproteases, which perform different functions in various cell types due to the fact that they are multidomain proteins. ADAM proteins act on a variety of substrates located in the plasma membrane to generate inflammatory, growth, migration and metabolic signals.

ADAMs 1-7 are expressed primarily in the reproductive organs and play an important role in spermatogenesis and egg-sperm fusion, although ADAM1, 4, 5 are also expressed in other tissues. ADAM9 is found in several tissues including the breast and lung and could play an important role in signal transduction. ADAM11 was identified after analysis of a locus for a presumed tumor suppressor gene. ADAM12 and ADAM19 are expressed in muscle tissue in embryonic and neonatal stages and in bones since the embryonic stage to adult life.

ADAM17, also known as the conversion enzyme of TNF-α (TACE), mediates the diffusion of TNF-α and its receptors (TNFRI and II), of the adhesion molecules (L-selectin, VCAM) and of different ligands of EGFR, such as amphiregulin, TGF-α and EGF-like growth factor that binds heparin (HB-EGF) [Blobel C.P. “Remarkable roles of proteolysis on and beyond the cell surface”. Curr Opin Cell Biol. 2000; 12: 606-12. Blobel C.P. “ADAMs: key components in EGFR signalling and development” Nat Rev Mol Cell Biol. 2005; 6: 32-43].

The latter class of molecules is involved in the development of inflammatory and fibrotic renal lesions in mice [Bollee G. et al. “Epidermal growth factor receptor promotes glomerular injury and renal failure in rapidly progressive crescentic glomerulonephritis”. Nat Med. 2011; 17: 1242-1250]. Recently, high serum concentrations of TNFRI and soluble solids have been shown to be a strong predictor of early renal function loss in both type 1 and type 2 diabetes [Gohda T. et al. “Circulating TNF receptors 1 and 2 predict stage 3 CKD in type 1 diabetes” J Am Soc Nephrol. 2012; 23: 516-24. Niewczas M. A. et al. J Am Soc Nephrol. “Circulating TNF receptors 1 and 2 predict ESRD in type 2 diabetes” 2012; 23: 507-15]. ADAM17 is also involved in the Notch cleavage in the plasma membrane to generate the Notch intracellular domain (NICD), which then moves to the nucleus to regulate gene expression [Murthy A. et al. “Notch activation by the metalloproteinase ADAM17 regulates myeloproliferation and atopic barrier immunity by suppressing epithelial cytokine synthesis” Immunity. 2012; 36: 105-19].

Notch is necessary for glomerular and proximal tubular development, its alteration is involved in diabetic nephropathy [Niranjan T. et al. “The Notch pathway in podocytes plays a role in the development of glomerular disease”. Nat Med. 2008; 14: 290-8].

The proteolytic activity of metalloproteases and proteins is finely regulated by endogenous inhibitors called TIMP (tissue inhibitor of metalloproteinases, 1/2/3/4) [Mohammed F. F. et al. “Metalloproteinases, inflammation, and rheumatoid arthritis”. Ann Rheum Dis. 2003; 62: ii43-7]. The TIMP3 tissue inhibitor is effective against most ADAM proteins, but represents, in the state of the art, the only known physiological inhibitor of ADAM17, and its reduction is associated with age-related renal fibrosis, tubulointerstitial fibrosis, important prognostic markers in a wide variety of renal diseases [Kawamoto H. et al. “Tissue inhibitor of metalloproteinase-3 plays important roles in the kidney following unilateral ureteral obstruction” Hypertens Res. 2006; 29: 285-94. Kassiri et al. “Loss of TIMP3 enhances interstitial nephritis and fibrosis” J Am Soc Nephrol. 2009; 20: 1223-35]. It has also been shown that the TIMP3 inhibitor blocks the binding of VEGF to the VEGF 2 receptor by inhibiting angiogenesis [Qi J.H. et al. “A novel function for tissue inhibitor of metalloproteinases-3 (TIMP3): inhibition of angiogenesis by blockage of VEGF binding to VEGF receptor-2” Nat Med. 2003; 9: 407-15], evidence is emerging that VEGF plays a crucial role in the maintenance of renal homeostasis, since the altered (increased or decreased) expression of VEGF leads to glomerular dysfunction and proteinuria [Rask-Madsen C., King G. L. “Kidney complications: factors that protect the diabetic vasculature”, Nat Med. 2010; 16: 40-1].

Furthermore, Notch and VEGF proteins intervene in podocytes of diabetic subjects where they are involved in the development of typical signs of diabetic nephropathy [Lin et al. “Modulation of notch-1 signaling alleviates vascular endothelial growth factor-mediated diabetic nephropathy” Diabetes. 2010; 59: 1915-25].

The direct correlation between the activation of the ADAM17 protein and the pathogenesis of diabetic nephropathy has been observed, and the deletion of its specific TIMP3 inhibitor contributes to the onset and progression of nephropathy in a mouse model of diabetes [Fiorentino L. et al. “Loss of TIMP3 underlies diabetic nephropathy via Fox01/STAT1 interplay”, EMBO Molecular Medicine. 2013; 5: 441-455]. The study showed that the expression of TIMP3, tissue inhibitor of metalloproteinase 3, is reduced in the kidney of diabetic mice compared to control mice, while the proteolytic activity of ADAM17 is increased. Timp3−/− diabetic mice have increased albuminuria and their kidneys have a higher degree of inflammation along with morphological and molecular alterations of the podocytes and increased basal membrane thickness compared to the wild type controls, indicating that the loss of TIMP3 is detrimental to the progression of the disease. The data of the gene expression of the kidneys of Timp3−/− diabetic mice was confirmed by the data deriving from the studies on renal biopsies obtained from patients with diabetic nephropathy, which showed a significant reduction of TIMP3 gene expression. The loss of TIMP3 is a hallmark of diabetic kidney disease in mouse models and in humans. Thus, TIMP3 plays an important role in the maintenance of renal homeostasis and represents an important therapeutic target for the control of diabetic nephropathy.

This observation makes the ADAM 17/TIMP3 system a possible new therapeutic objective for diabetic nephropathy. The therapies currently used to counter diabetic nephropathy, such as blood glucose control, angiotensin II receptor blockers (ATII) and ACE inhibitors, slow down, but do not stop, the progression of this disease [Ruggenenti P. et al. “The RAAS in the pathogenesis and treatment of diabetic nephropathy”. Nat Rev Nephrol. 2010; 6: 319-30]. Nonetheless, on the therapeutic scene no drug is specific for renal disease in diabetics.

Furthermore, promoting a targeted and specific delivery mechanism of a drug, of a protein or peptide nature, to the kidney appears to be an attractive method to increase the effectiveness of treatment based on this drug, improving the therapeutic index and the pharmacokinetic profile. Specifically, a targeted transport of an ADAM17 protein inhibitor could ensure optimal enzymatic inhibition, particularly in the kidney, without side effects in other districts.

There is currently no specific therapy for the treatment of diabetic nephropathy. The subjects suffering from this condition use therapies directed to the metabolic and pressure control, able to contrast the clinical signs of the disease, but do not take any “biological” drug, that is, aimed at a specific molecular mechanism at the base of the pathology.

### SUMMARY OF THE INVENTION

The kinetic analysis of the TIMP3 protein showed that all the critical elements necessary for the inhibition of ADAM17 reside in the N-terminal domain of the tissue inhibitor of metalloprotease 3 (TIMP-3) [Meng-Huee L. et al. “Mapping and characterization of the functional epitopes of tissue inhibitor of metalloproteinases (TIMP)-3 using TIMP-1 as the scaffold: A new frontier in TIMP engineering” Protein Science. 2002; 11: 2493-2503]. Furthermore, the kinetic inhibition studies for MMP and ADAM17 have shown that the T2G mutation of N-TIMP-3 is a potent inhibitor of ADAM17 but is an extremely weak inhibitor of MMPs (Shuo Wei et al. “Reactive Site Mutations in Tissue Inhibitor of Metalloproteinase-3 Disrupt Inhibition of Matrix Metalloproteinases but Not Tumor Necrosis Factor-α-converting Enzyme”. J. Biol. Chem. 2005; 280: 32877-32882]. Starting from these elements, the present invention provides fusion peptides consisting of the peptide fragment corresponding to the N-terminal domain derived from the human TIMP3 protein, both in native and mutated form, bound by the N-terminal end to a highly selective and efficient carrier peptide for transport in renal proximal tubule cells.

A second object of the invention is the medical use of fusion peptides consisting of a peptide fragment derived from the tissue inhibitor human protein of metalloproteinase 3, TIMP3, both in native and mutated form, bound to the N-terminal end to a carrier peptide specific for renal proximal tubule cells.

A further object of the invention is the use of said fusion peptides in the treatment of diabetic nephropathy.

Also included in the scope of the present invention are the compositions comprising said melting peptides and the medical use thereof, in particular the use thereof in the treatment of diabetic nephropathy.

### BRIEF DESCRIPTION OF THE FIGURES

FIG. 1 is the graph representing the blood glucose concentration in animals after 8 weeks of treatment with the peptide according to the invention G3-C12-T2GNTIMP3 having SEQ ID No. 4.

FIG. 2 is the graph representing the total albumin concentration in the collected urine of the 24 hours prior to the sacrifice in animals after 8 weeks of treatment with the peptide according to the invention G3-C12-T2GNTIMP3 having SEQ ID No. 4.

FIG. 3 presents the graphs showing the glomerular structure analysis by PAS staining of renal sections expressed as a function of the middle glomerular area (A), of the mesangial area (B) and of the mesangial fraction area (C) in animals after 8 weeks of treatment with the peptide according to the invention G3-C12-T2GNTIMP3 having SEQ ID No. 4.

FIG. 4 shows the analysis of the tissue expression of fibrosis markers respectively collagen IV (A), fibronectin (B) and NOX4 (C) in animals after 8 weeks of treatment with the peptide according to the invention G3-C12-T2GNTIMP3 having SEQ ID No. 4.

FIG. 5 shows the analysis of the expression of podocin in renal cortex extracts in animals after 8 weeks of treatment with the peptide according to the invention G3-C12-T2GNTIMP3 having SEQ ID No. 4.

### DETAILED DESCRIPTION OF THE INVENTION

The present invention describes a fusion peptide consisting of a peptide fragment derived from the tissue inhibitor human protein of metalloproteinase 3, TIMP3, bound at its N-terminal end to a carrier peptide, highly selective for renal proximal tubule cells.

The terms “peptide”, “peptide fragment” and “polypeptide” are used in the present description as synonyms and, unless otherwise specified, refer to polymeric nitrogen organic compounds resulting from the combination of two or more amino acids bound by peptide bonds, deriving from the decomposition of proteins. The term also includes oligopeptides, protein fragments, analogues and protein derivatives, pegylated derivatives, glycosylated derivatives, acylated derivatives and the like commonly understood by those skilled in the art.

According to the present invention, in the peptide each amino acid residue may be present as a configurational isomer (D)- or (L)-, in so far as the peptide maintains its functional properties.

The tissue inhibitor of metalloproteinase 3 is encoded in the human species by the TIMP3 gene [Apte S. S. et al. “Cloning of the cDNA encoding human tissue inhibitor of metalloproteinases-3 (TIMP-3) and mapping of the TIMP3 gene to chromosome 22” Genomics. 1994; 19: 86-90; Qi J. H., et al. “A novel function for tissue inhibitor of metalloproteinases-3 (TIMP3): inhibition of angiogenesis by blockage of VEGF binding to VEGF receptor-2”. Nat Med. 2003; 9: 407-15].

TIMP3 is the most expressed TIMP in the kidney [Catania J. M. et al. “Role of matrix metalloproteinases in renal pathophysiologies”. Am J Physiol Renal Physiol. 2007; 292: F905-11] and has a broad protease inhibition profile. Its reduction in mouse models of diabetic nephropathy is involved in inflammation processes, renal fibrosis and tubular interstitial lesions [Ford B.M. et al. “ADAM17 mediates Nox4 expression and NADPH oxidase activity in the kidney cortex of OVE26 mice”. Am J Physiol Renal Physiol. 2013; 305: F323-32; Kassiri Z. et al. “Loss of TIMP3 enhances interstitial nephritis and fibrosis” J Am Soc Nephrol. 2009; 20: 1223-35] and is associated with an increase in mesangial expansion and microalbuminuria [Basu R. et al. “Loss of TIMP3 selectively exacerbates diabetic nephropathy” Am J Physiol Renal Physiol. 2012; 303: F1341-52]. TIMP3 is the only known physiological inhibitor of ADAM17, the metalloprotease responsible for the activation of several ligands involved in the pathogenesis of chronic kidney disease and glomerulonephritis [Bollee G. et al. “Epidermal growth factor receptor promotes glomerular injury and renal failure in rapidly progressive crescent glomerulonephritis” Nat Med. 2011; 17: 1242-50].

The expression and activity of ADAM17 were found in the renal cortex of mouse models of type 1 diabetes and in renal cells exposed to high glucose concentrations [Ford B. M. 2013]. The high plasma concentration of two ADAM17 substrates, such as TNFR1 and TNFR2, has recently been associated with phase 3 of chronic kidney disease in patients with type 1 and type 2 diabetes [Niewczas M. A. et al. “Circulating TNF receptors 1 and 2 predict ESRD in type 2 diabetes”. J Am Soc Nephrol. 2012; 23: 507-15; Gohda T. et al. “Circulating TNF receptors 1 and 2 predict stage 3 CKD in type 1 diabetes” J Am Soc Nephrol. 2012; 23: 516-24]. Furthermore, the role for ADAM17 as a mediator of the angiotensin II (Angio) profibration effect has been demonstrated [Chodavarapu H. “Rosiglitazone treatment of type 2 diabetic db/db mice attenuates urinary albumin and angiotensin converting enzyme 2 excretion”. PLoS One. 2013; 8: e62833]. Recent studies have shown increased urinary ACE2 activity associated with increased renal protein expression of ACE2 and ADAM17 and progression of renal damage in diabetic nephropathy [Salem E. S. “Insulin treatment attenuates renal ADAM17 and ACE2 shedding in diabetic Akita mice”. Am J Physiol Renal Physiol. 2014; 306: F629-39].

The design of the peptide fragments of the TIMP3 protein used in the present invention began with the analysis of the human protein [Douglas D. A. et al. “Computational Sequence Analysis of the Tissue Inhibitor of Metalloproteinase Family” Journal of Protein Chemistry. 1997, 16: 237-255].

The analysis of the inhibition kinetics showed that all the elements necessary for the inhibition of the ADAM 17 protein reside in the N-terminal domain of the TIMP-3 molecule [Lee M. H. et al. Mapping and characterization of the functional epitopes of tissue inhibitor of metalloproteinases (TIMP)-3 using TIMP-1 as the scaffold: A new frontier in TIMP engineering” Protein science 2002, 11: 2493-2503].

According to the invention, the selected portion of the human TIMP3 protein used to produce the fusion peptide corresponds to the N-terminal portion of the protein constituting the loop 1 (aa 24-143). The protein sequence placed upstream of the selected fragment, consisting of its first 23 amino acids (aa 1-23), without any element having an inhibitory activity, has been substituted in the fusion peptide according to the present invention with an amino acid sequence having the function of a carrier peptide.

According to the present invention, the carrier peptide used in the formation of the fusion peptide is the peptide G3-C12, having Seq. ID No. 1: ANTPCGPYTHDCPVKR, ligand of the galectin-3 protein and identified as a highly selective and efficient transporter for renal proximal tubule cells.

Animal testing has shown that the G3-C12 peptide is a vector that can accumulate in a highly specific manner in the murine kidneys after intravenous injection; also in conjugation with drugs, it shows high selectivity and rapid renal accumulation, renal clearance in a few hours and without toxicity: it has been successfully used in conjugation with the angiotensin-converting enzyme (ACE) inhibitor, captopril [Geng Q. et al. “Peptide-drug conjugate linked via a disulfide bond for kidney targeted drug delivery”. Bioconjug Chem. 2012; 23: 1200-10].

The conjugation of the carrier G3-C12 fragment with the peptide fragment of the human TIMP3 protein, or with variants thereof, as described below, represents a valid approach to obtain a high level of expression of the inhibitor of the ADAM17 activity directly in the kidney through reabsorption in proximal tubular cells, in all those pathologies, such as diabetic nephropathy, characterized by a reduction in the renal expression of TIMP3.

The specific transport of the G3-G12-mediated peptide to the renal district greatly increases the efficacy of the active ingredient, improving the therapeutic index and pharmacokinetic profile thereof.

In an embodiment of the invention, the amino acid sequence of the peptide fragment (aa 24-143) of the human TIMP3 protein used exactly coincides with the (wt) native sequence of the corresponding human protein region and has Seq. ID No. 2: MCTCSPSHPQDAFCNSDIVIRAKVVGKKLVKEGPFGTLVYTIKQMKMYRGFTKMPHVQYIHTEASE SLCGLKLEVNKYQYLLTGRVYDGKMYTGLCNFVERWDQLTLSQRKGLNYRYHLGCK. According to the invention, the peptide fragment (aa 24-143) of the human TIMP3 protein is conjugated at its N-terminal end to the amino acid carrier sequence of the G3-C12 carrier peptide. The fusion peptide thus obtained has SEQ. ID No. 3:

ANTPCGPYTHDCPVKRMCTCSPSHPQDAFCNSDIVIRAKVVGKKLVKEG
PFGTLVYTIKQMKMYRGFTKMPHVQYIHTEASESLCGLKLEVNKYQYLL
TGRVYDGKMYTGLCNFVERWDQLTLSQRKGLNYRYHLGCK.

Alternatively, in other embodiments of the invention the peptide fragment of the TIMP3 protein may also have a modified amino acid sequence, i.e. mutated with respect to the native sequence, in which the mutation may be point-like, i.e. affecting a single amino acid, for example, substitutions, insertions and deletions, or involving at least two amino acid residues in succession.

In a particularly preferred embodiment of the invention, the fusion peptide, instead of exhibiting the native amino acid sequence of the peptide fragment, is composed of the N-terminal portion of the human TIMP3 protein (aa 24-143) constituting the protein loop 1, mutated by substitution of the amino acid threonine (Thr) in position 2 with a glycine Gly (T2G) (T2G-N-TIMP3) and conjugated at its N-terminal end to the amino acid carrier sequence of the G3-C12 carrier peptide.

The substitution of the threonine amino acid with glycine causes the removal of the side chain of the residue 2, thus considerably reducing the affinity for MMP-1, -2 and -3. A peptide fragment (T2G-N-TIMP3) is thus obtained with an effective inhibition action of the ADAM17 protein, but extremely weak against the four main metalloproteases (collagenase 1, gelatinase A, stromelysin 1 and membrane type 1 MMP) which, combined with the carrier peptide G3-C12, produces the fusion peptide having sequence SEQ ID No. 4:

ANTPCGPYTHDCPVKRMCGCSPSHPQDAFCNSDIVIRAKVVGKKLVKEG
PFGTLVYTIKQMKMYRGFTKMPHVQYIHTEASESLCGLKLEVNKYQYLL
TGRVYDGKMYTGLCNFVERWDQLTLSQRKGLNYRYHLGCK.

In a further alternative embodiment of the present invention, the fusion peptide derives from the combination of the carrier peptide G3-C12 with a peptide derived from the human TIMP3 protein characterized by the addition (mutation by insertion) of an N-terminal alanine residue (-1A) (1A-NTIMP3). The insertion of the alanine residue at the N-terminal end of the fragment causes the alteration of the interaction of the cysteine residue in position 1 with the active site of the metalloprotease, with consequent drastic reduction of its activity; the fusion peptide having SEQ ID No. 5:

ANTPCGPYTHDCPVKRMACTCSPSHPQDAFCNSDIVIRAKVVGKKLVKE
GPFGTLVYTIKQMKMYRGFTKMPHVQYIHTEASESLCGLKLEVNKYQYL
LTGRVYDGKMYTGLCNFVERWDQLTLSQRKGLNYRYHLGCK
(G3-C12-1A-NTIMP3) is thus obtained.

In another embodiment of the invention, in the fusion peptide, the peptide fragment of the NTIMP3 protein, or variants thereof, instead of being conjugated to the carrier peptide G3-C12, is conjugated to another peptide sequence, also identified as excellent specific carrier of the renal district, the peptide (KKEEE)₃K having Seq. ID No. 6: KKEEEKKEEEKKEEEK.

According to the invention, therefore, the following are obtained:

the fusion peptide (KKEEE)₃K-NTIMP3 having SEQ. ID No. 7:

KKEEEKKEEEKKEEEKMCTCSPSHPQDAFCNSDIVIRAKVVGKKLVKEG
PFGTLVYTIKQMKMYRGFTKMPHVQYIHTEASESLCGLKLEVNKYQYLL
TGRVYDGKMYTGLCNFVERWDQLTLSQRKGLNYRYHLGCK;

the fusion peptide (KKEEE)₃K-T2GNTIMP3 having SEQ. ID No. 8:

KKEEEKKEEEKKEEEKMCGCSPSHPQDAFCNSDIVIRAKVVGKKLVKEG
PFGTLVYTIKQMKMYRGFTKMPHVQYIHTEASESLCGLKLEVNKYQYLL
TGRVYDGKMYTGLCNFVERWDQLTLSQRKGLNYRYHLGCK;

and

the fusion peptide (KKEEE)₃K-1A-NTIMP3 having SEQ. ID No. 9:

KKEEEKKEEEKKEEEKMACTCSPSHPQDAFCNSDIVIRAKVVGKKLVKE
GPFGTLVYTIKQMKMYRGFTKMPHVQYIHTEASESLCGLKLEVNKYQYL
LTGRVYDGKMYTGLCNFVERWDQLTLSQRKGLNYRYHLGCK.

The peptide according to the invention is obtained by automatic synthesis in solid phase with a purity higher than 98%, analyzed by HPLC and mass spectrometry, according to the techniques known to those skilled in the art, who however will easily understand that, as an alternative to the chemical synthesis method, the peptides may be obtained by recombinant techniques and fermentation in bacterial cells of E. coli DH5alpha, through the use of plasmids containing the gene sequences coding for the specific peptide fragments [Sambrock et al. 1989, 1992, 2001, Molecular Cloning: A laboratory Manual, Cold Spring Harbour Laboratory, New York].

The fusion peptides according to the invention may be used as such, as a pharmaceutical active ingredient, or together with other active ingredients having therapeutic activity, and may form part of pharmaceutical compositions which comprise them.

In a particularly preferred embodiment of the invention, the peptide according to the invention, alone or in combination with other active ingredients, constitutes the active ingredient of pharmaceutical compositions comprising said peptide and at least one pharmaceutically acceptable carrier.

Said pharmaceutical composition may further comprise: solvents, stabilizers and excipients known to those skilled in the art, such as aqueous solution, buffered physiological saline, polyethylene glycol, stabilizing agents, antioxidants and other compounds widely known to those skilled in the pharmaceutical technique.

The pharmaceutical composition of the present invention is in pharmaceutical form suitable for being parenterally, intravenously, intramuscularly, subcutaneously and intraperitoneally administered.

Previous studies by the Applicant have shown that the loss of TIMP3 function contributes to the onset and progression of diabetic kidney disease in human patients and in mouse models of diabetes. This led to the hypothesis that in vivo manipulation of the biological activity regulated by the TIMP3/ADAM17 interaction in rodent models may limit the onset and/or progression of diabetic renal complications. Preliminary results obtained have suggested that the regulation of ADAM 17 protein activity may represent a valid therapeutic target, in particular for diabetic renal nephropathy. It is worth mentioning that at present no inhibitors are available for the ADAM17 protein, nor are drugs specifically directed against diabetic nephropathy.

As demonstrated by the experimentation presented below, the fusion peptide and the compositions comprising it have protective activities at the renal level in a context of chronic hyperglycemia, and are therefore particularly useful in the treatment of renal diseases, in particular in the treatment of diabetic nephropathy.

Experimental Part

The invention will now be illustrated with reference to examples and methods of use and experimental tests which do not limit the scope of application of the invention.

### EXAMPLE 1

### Synthesis and Purification of Peptides

All the peptides used in the experimentation aimed at demonstrating the effectiveness and the advantage deriving from the use of the invention were produced by chemical synthesis from the C-terminal end to the N-terminal end by ProteoGenix SAS (France).

The peptides thus produced were purified using preparative HPLC (Kromasil 100-5) (50 mg, purity>95%). The quality of the peptides was analyzed by HPLC (LC3000) and mass spectrometry (Shimadzu LCMS-2020) according to the standard techniques widely known to those skilled in the art.

### EXAMPLE 2

### Transport to the Kidney in a Mouse Model of Diabetic Nephropathy

All procedures performed on mice have been approved by the University Committee for the care and use of animals at the University “Tor Vergata”. The animals are fed a standard diet for rodents and water ad libitum and kept in sterile cages (5 mice per cage) in a plant with a light-dark cycle of 12-12 hours. The DBA/2J mice are obtained from Jackson Laboratory (Maine, USA); only male mice are used in the experiment because the females are genetically protected against diabetes and kidney anomalies are mild. Mice are rendered diabetic at 8 weeks of age with a protocol that provides for the low dose administration of streptozotocin (STZ), a compound that has a preferential toxicity to pancreatic cells [symbol] .

In short, six-week-old mice receive sodium citrate (control) or STZ (45 mg/kg, pH 4.5, dissolved in sodium citrate) by intraperitoneal injection for 5 consecutive days. One week after the first administration of STZ, using an automated Onetouch Lifescan glucometer (Milpitas, Calif.), fasting glucose levels (4h) are measured; mice with a fasting glucose level above 250 mg/dL for 2 consecutive days are considered diabetic and used for this study.

Four weeks after the onset of diabetes, the rats are randomly divided into different groups (n=10 mice per group):

Group 1 consisting of mice that received PBS as a control, Group 2 consisting of mice that received the G3-C12-NTIMP3 fusion peptide,

Group 3 consisting of mice that received the G3-C12-T2GNTIMP3 fusion peptide,

Group 4 consisting of mice that received the G3-C12-1A-NTIMP3 fusion peptide,

Group 5 consisting of mice that received the fusion peptide (KKEEE)₃K-NTIMP3,

Group 6 consisting of mice that received the fusion peptide (KKEEE)₃K-T2GNTIMP3,

Group 7 consisting of mice that received the fusion peptide (KKEEE)₃K-1A-NTIMP3.

All mice receive the amount of the respective peptide equal to 2 mg/kg of weight diluted in 100 [symbol] l of saline solution by ip injection for 8 weeks, 2 times a week. After 8 weeks of treatment, the mice are sacrificed. Prior to sacrifice, mice are placed in metabolic cages for 24-hour urine collection and urine albuminuria determination and blood sample collection. The level of albumin in the urine collected in the 24 hours before the sacrifice is determined using an Elisa kit specific for the determination of murine albumin (Abcam) used according to the instructions provided.

Results

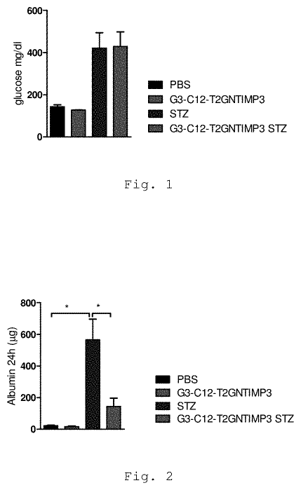

At the end of the 8 weeks of treatment in all animals, the blood glucose concentration was evaluated by analysis of a drop of blood obtained by ocular sampling [Onetouch Lifescan (Milpitas, Calif.)]. The result obtained shown in FIG. 1 indicates that treatment with the peptide does not act on the glycemic levels, in fact all the animals injected with STZ, regardless of the subsequent treatment, show a high concentration of blood glucose, unlike the controls.

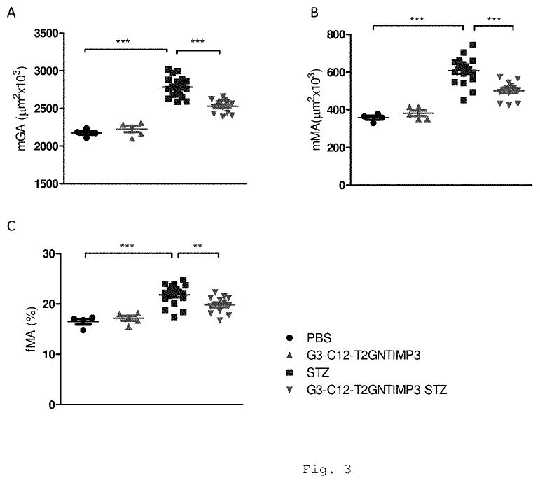

The results of albuminuria measurement in animals of the various experimental groups shown in FIG. 2 indicate that in diabetic animals (STZ), the loss of albumin with urine exceeding the physiological limit, early marker of renal damage, is significantly increased compared to control animals (PBS), while peptide treatment induces a significant reduction of 24-hour urinary albumin in diabetic animals (STZ+G3C12-T2GNTIMP3) compared to diabetic animals treated with PB (STZ) (*p<0.05. Student's t test, the data means±SEM).

### Example 3

### Morphological and Immunohistochemical Analysis of the Kidney

At the end of the treatment and of the physiological in vivo detections, the kidneys were taken from the sacrificed animals and the histological analysis and the quantification of the lesions was performed on them as already described in Fiorentino L. et al. The mean glomerular area (mGA), the mean mesangial area (mMA) and the mesangial area fraction (fMA) are also evaluated. In addition, fibrosis markers (Collagen IV, Fibronectin, αSMA, TGFβ), inflammation (F4/80, MCP1), podocyte damage (WT1, nephrine, podocin, NOTCH), EMT/EndoMT (N-cadherin, VE-cadherin, Vimentin), and oxidative stress (CML, NOX4, Nitrotyrosine) are evaluated by immunohistochemistry and by Western blot analysis and analysis of gene expression by qRT-PCR, on protein extract and on total RNA isolated from the renal cortex, respectively.

Results

The results of the glomerular structure analysis by PAS staining of the renal sections shown in FIG. 3 show a significant reduction in the average glomerular area in (A), of the average mesangial area in (B), and of the mesangial fraction area in (C), of the diabetic animals treated with the peptide according to the invention and having Seq. ID. 4 compared to the untreated ones, suggesting a significant protective effect determined by treatment with the fusion peptide (***p<0.0001; **p<0.01. Student's t test, the data means±SEM).

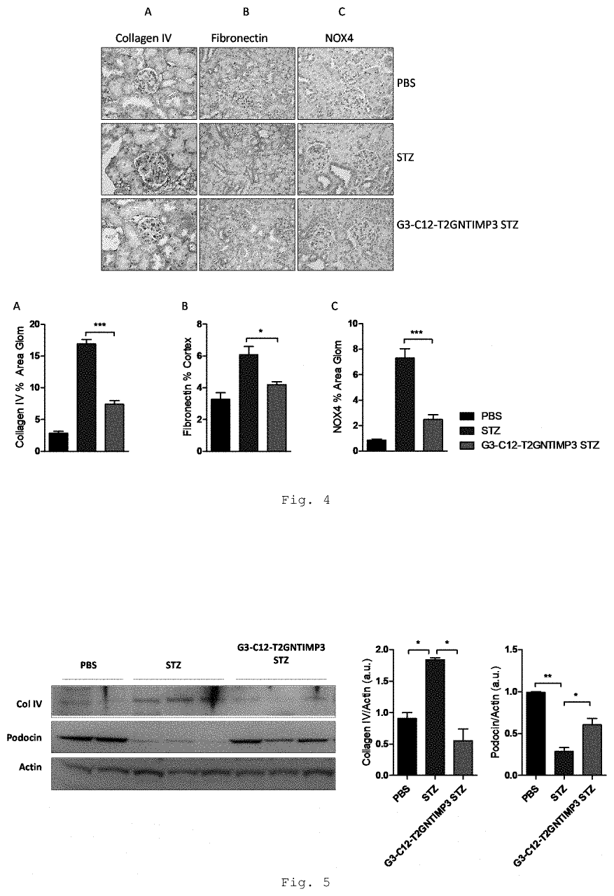

As for the fibrosis markers analyzed, FIG. 4 shows that at the end of the 8 weeks of treatment with the G3-C12-T2GNTIMP3 fusion peptide in all animals, the tissue expression of Collagen IV (A) (antibody used with dilution 1:700, Abcam) and Fibronectin (B) (antibody used with dilution 1:300, Sigma) and oxidative stress NOX4 (C) (antibody used with dilution 1:200, Abcam) detected by histochemical staining is significantly reduced compared to diabetic animals, in which, on the contrary, increased levels of these indices are evident compared to those found in the non-diabetic mouse (control that received PBS) (***p<0.0001; *p<0.05. Student's t test, the data means±SEM).

FIG. 5 shows that the treatment with the fusion peptide G3-C12-T2GNTIMP3 is capable of restoring the expression level of podocin, an index of podocyte function, to a level comparable to that of the non-diabetic mouse (control that received PBS), which is instead reduced in the diabetic condition. On the contrary, the expression of Collagen IV, index of fibrosis, is reduced following treatment with G3-C12-T2GNTIMP3 in diabetic animals compared to diabetic controls treated with PBS alone (**p<0.005; *p<0.05. Student's t test, the data means±SEM).

Overall, the results obtained indicate that the conjugation of G3-C12 with T2G-N-Timp3 represents a valid approach to obtain a high level of expression of the inhibitor of ADAM17 activity directly in the kidney through reabsorption in proximal tubular cells, confirming an important protective role of the G3-C12-T2GNTIMP3 peptide in the treatment of diabetic nephropathy.

Statistical Snalysis of Data

The results of the experimental studies conducted are expressed as mean values±SD. Depending on the sample distribution, the statistical comparison for molecular analysis is performed using the non-parametric Student's t-test for independent samples or non-parametric Mann-Whitney comparisons to verify a priori hypotheses of differences between two groups. Similarly, ANOVA is used for the analysis and comparison of appropriate post-hoc parameters (for example, ordinary one-way ANOVA with Bonferroni, Tukey or Holm-Sidak test for multiple comparisons) or non-parametric (i.e. Kruskal-Wallis test with Dunn test for multiple comparisons). Linear correlation analysis is performed using Spearman's test or Pearson's test based on sample distribution. The values of p<0.05 are considered statistically significant. All analyses are performed with GraphPad Prism 6.0 (GraphPad, San Diego, Calif., USA) which will eventually adapt the test using different algorithms depending on the sample distribution.

Conclusion

Diabetic nephropathy, a condition for which there are currently no specific and effective pharmacological treatments, is an important cause of end-stage renal disease characterized by albuminuria and progressive decline of renal function. At the histopathological level, diabetic nephropathy is characterized by glomerular hypertrophy (thickening of the glomerular basement membrane), glomerular hypertrophy and tubulointerstitial lesions that overall contribute to the decline of the renal function. Overall, both glomerulosclerosis and interstitial fibrosis are part of the process that leads to a decline in renal function in diabetes. It is known that TIMP3 and ADAM17 proteins play a role in both glomerulosclerosis (TIMP3 and ADAM17) and interstitial fibrosis (ADAM17).

The present description demonstrates that with the present invention, a valid active ingredient is provided for the treatment of diabetic nephropathy which consists in the development of different fusion peptides capable of restoring the inhibitory effect of the TIMP3 protein on ADAM17, reducing some of the typical signs of the pathology. Preliminary data obtained from the experimentation carried out to prove the efficacy of the invention demonstrate a significant and consistent decrease of albuminuria together with a glomerulosclerosis and an improved tubulointerstitial fibrosis following treatment with the peptides according to the invention described herein.

1. A fusion peptide consisting of:
(i) a peptide fragment of the human tissue inhibitor of metalloproteinase 3, TIMP3, corresponding to the N-terminal amino acid portion of said protein (aa 23-143), or a modified form of said peptide fragment,
(ii) a peptide carrier selective for renal proximal tubule cells, or a modified form of said carrier peptide,
wherein the fragment of the TIMP3 protein is bound at its N-terminal end to the carrier peptide.

2. The fusion peptide according to claim 1 wherein the peptide carrier selective for renal proximal tubule cells is the G3-C12 peptide having Seq. ID. No. 1:
ANTPCGPYTHDCPVKR.

3. The fusion peptide according to claim 1, wherein the peptide carrier selective for renal proximal tubule cells is the peptide (KKEEE)3K having Seq. ID. No. 6:
KKEEEKKEEEKKEEEK.

4. The fusion peptide according to claim 3, wherein the amino acid sequence of the peptide fragment of the tissue inhibitor human protein of the metalloproteinase 3, TIMP3, corresponding to the N-terminal portion of said protein (aa 23-143) exactly coincides with the native protein sequence having Seq. ID. No. 2.

5. The fusion peptide according to claim 2, wherein the amino acid sequence of the peptide fragment derived from the tissue inhibitor human protein of the metalloproteinase 3, TIMP3, corresponding to the N-terminal portion of said protein (aa 23-143) presents point mutations with respect to the native protein sequence, affecting at least two amino acid residues in succession.

6. The fusion peptide according to claim 5 wherein the position 2 of the amino acid sequence of the peptide fragment derived from the tissue inhibitor human protein of the metalloproteinase 3, TIMP3, and corresponding to the N-terminal portion of said protein (aa 23-143) is mutated by substitution of the threonine amino acid with a glycine (T2G-N-TIMP3).

7. The fusion peptide according to claim 5, wherein the amino acid sequence of the peptide fragment derived from the tissue inhibitor human protein of metalloproteinase 3, TIMP3, corresponding to the N-terminal portion of said protein (aa 23-143) is mutated by insertion of an alanine residue at position -1, i.e. upstream of the TIMP3 fragment sequence.

8. The fusion peptide according to claim 4 having SEQ. ID No. 3.

9. The fusion peptide according to claim 6 having SEQ. ID No. 4.

10. The fusion peptide according to claim 7 having SEQ. ID No. 5.

11. The fusion peptide according to claim 4 having SEQ. ID No. 7.

12. The fusion peptide according to claim 6 having SEQ. ID No. 8.

13. The fusion peptide according to claim 7 having SEQ. ID No. 9.

14. The fusion peptide according to claim 1, wherein each amino acid residue may be present both as a (D)- and (L)-configurational isomer.

15. The fusion peptide according to claim 1, wherein the modified forms of the two peptide fragments are pegylated, glycosylated, acylated analogs of the native peptide fragments.

16. A method of medical treatment comprising providing the fusion peptide as defined in claim 1, and administering an effective amount of the fusion peptide.

17. A method for treatment of diabetic nephropathy, comprising providing the fusion peptide of claim 1, and administering an effective amount of the fusion peptide.

18. A composition comprising the fusion peptide as defined in claim 1 and at least one pharmaceutically acceptable carrier.

19. The composition according to claim 18 further comprising solvents, stabilizers, buffering agents, antioxidants.

20. A composition according to claim 19 in a pharmaceutical form suitable for administering parenterally, intravenously, intramuscularly, subcutaneously and intraperitoneally administered.

21. A method of medical treatment comprising providing the composition of claim 20, and administering an effective amount of the composition.

22. A method for treatment of diabetic nephropathy, comprising providing the composition of claim 20, and administering an effective amount of the composition.
